(19) United States
(12) Patent Application Publication
(10) Pub. No.: US 2021/0007574 A1
(43) Pub. Date: Jan. 14, 2021

(54) MEDICAL DEVICE MANAGEMENT SYSTEM AND MEDICAL DEVICE MANAGEMENT METHOD

(71) Applicant: SONY CORPORATION, TOKYO (JP)

(72) Inventors: Tomoyuki HIRAYAMA, KANAGAWA (JP); Tsuneo HAYASHI, TOKYO (JP); Yuki SUGIE, KANAGAWA (JP)

(21) Appl. No.: 16/978,296

(22) PCT Filed: Feb. 28, 2019
(86) PCT No.: PCT/JP2019/007779

§ 371 Date: Sep. 4, 2020

(30) Foreign Application Priority Data
Mar. 13, 2018 (JP) 2018-045175

(51) Int. Cl. A61B 1/00; A61B 1/06; A61B 1/04

(52) U.S. Cl. CPC A61B 1/00009; A61B 1/0661; A61B 1/00057; A61B 1/00045; A61B 1/042

(57) ABSTRACT

[Overview] [Problems to be Solved] To perform optimization of each of a plurality of surgical devices in consideration of the plurality of surgical devices. [Solution] A medical device management system is provided that includes a plurality of devices connected to a surgical network, and an information processor configured to change settings of one or more devices out of the plurality of devices via the surgical network. The information processor acquires two or more pieces of information related to the plurality of devices and changes at least one of the settings of the plurality of devices on the basis of the two or more pieces of information.

### TECHNICAL FIELD

The present disclosure relates to a medical device management system and a medical device management method.

### BACKGROUND ART

In recent years, various devices have been provided in an operating room. This leads to a disadvantage that it is necessary to set each of the devices for each operation and thus efficiency of surgery is reduced due to time taken for settings. Under the circumstances, as described in PTL 1 listed below, for example, a technique is proposed that allows settings prepared in advance to be read into a plurality of devices from patient information to thereby shorten the time for setting of the devices.

### CITATION LIST

### Patent Literature

PTL 1: Japanese Unexamined Patent Application Publication No. 2002-336268

### SUMMARY OF THE INVENTION

### Problems to be Solved by the Invention

However, the technique described in PTL 1 above only allows for individual settings of each device, and it is thus difficult with this technique to perform device optimization in consideration of a plurality of devices present in an operating room.

Hence, it is desired to perform optimization of each of a plurality of surgical devices in consideration of the plurality of surgical devices.

### Means for Solving the Problems

According to the present disclosure, there is provided a medical device management system including a plurality of devices connected to a surgical network and an information processor configured to change settings of one or more devices out of the plurality of devices via the surgical network. The information processor acquires two or more pieces of information related to the plurality of devices and changes at least one of the settings of the plurality of devices on the basis of the two or more pieces of information.

In addition, according to the present disclosure, there is provided a medical device management method including a plurality of devices connected to a surgical network, and changing settings of one or more devices out of the plurality of devices connected to the surgical network. The method acquires two or more pieces of information related to the plurality of devices and changes at least one of the settings of the plurality of devices on the basis of the two or more pieces of information.

### Effect of the Invention

As described above, according to the present disclosure, it becomes possible to perform optimization of each of a plurality of surgical devices in consideration of the plurality of surgical devices. This makes it possible to shorten the time for setting of the devices and thereby allows improvement of efficiency of surgery.

It is to be noted that the effect described above is not necessarily limiting, and along with or instead of the above-described effect, any effect that is illustrated in the present specification or other effects that may be expected from the present specification may be exhibited.

### BRIEF DESCRIPTION OF DRAWINGS

FIG. 1 is a schematic diagram illustrating a configuration of an endoscopic surgical system.

FIG. 2 is a schematic diagram illustrating a surgical system connected to an IP network.

FIG. 3 is a schematic diagram illustrating a configuration example in which in a case where each of devices illustrated in FIG. 2 does not have an interface of the IP network, an IP converter performs interface conversion between that device and the IP network.

FIG. 4 is a schematic diagram illustrating a configuration of the IP converter.

FIG. 5 is a flowchart illustrating basic processing of the system.

FIG. 6 is a flowchart illustrating processing from installation of each of the devices of the surgical system to completion of adjustments of the devices.

FIG. 7 is a schematic diagram illustrating a surgical system in which a tablet terminal is added to the surgical system of FIG. 2.

FIG. 8 is a schematic diagram illustrating a transition on a display screen of the tablet terminal.

FIG. 9 is a schematic diagram illustrating how a monitor is adjusted in the configuration of the surgical system illustrated in FIG. 3.

FIG. 10 is a flowchart illustrating processing related to adjustments of the monitor.

FIG. 11 is a schematic diagram illustrating how an endoscope system is adjusted in the configuration of the surgical system illustrated in FIG. 3.

FIG. 12 is a flowchart illustrating processing related to adjustments of the endoscope system.

FIG. 13 is a schematic diagram illustrating how a light source is adjusted in the configuration of the surgical system illustrated in FIG. 3.

FIG. 14 is a flowchart illustrating processing related to adjustments of the light source.

FIG. 15 is a diagram illustrating processing of system adjustments in a case where an error remains in the adjustment of the monitor, the adjustment of the endoscope system, and the adjustment of the light source.

FIG. 16 is a flowchart illustrating processing of system adjustments in a case where the error remains.

FIG. 17 is a flowchart illustrating processing related to a second embodiment.

FIG. 18 is a schematic diagram illustrating adjustment spots.

FIG. 19 is a schematic diagram illustrating an extended surgical system.

### MODES FOR CARRYING OUT THE INVENTION

In the following, preferred embodiments of the present disclosure are described in detail with reference to the drawings. It is to be noted that, in the present specification and the drawings, components that have substantially the same functions and configurations are denoted with the same reference signs, and redundant descriptions are omitted.

It is to be noted that the description is given in the following order.

1. Overview of Surgical System

2. Overview of Surgical System According to Present Disclosure

3. Configuration Example of Surgical System

4. First Embodiment
4.1. Adjustment of Monitor
4.2. Adjustment of Endoscope System
4.3. Adjustment of Light Source
4.4 Case Where Error Remains in Each Adjustment

5. Second Embodiment

6. Third Embodiment

7. Fourth Embodiment

8. Fifth Embodiment

9. Sixth Embodiment

10. Seventh Embodiment

### 1. Overview of Surgical System

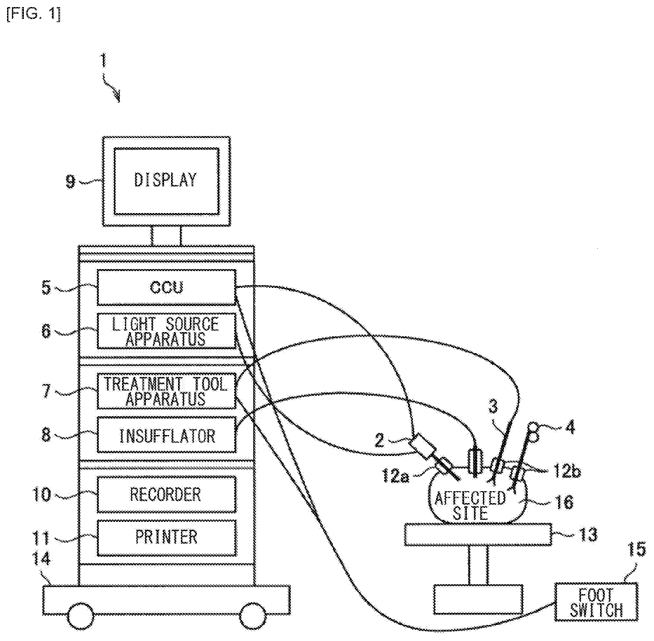

In recent years, endoscopic surgery has been carried out in medical practice instead of traditional laparotomy. For example, in a case where abdominal surgery is performed, an endoscope surgical system 1 placed in an operating room as illustrated in FIG. 1 is used. Instead of making an incision in an abdominal wall to open the abdomen as in a traditional method, opening instruments referred to as trocars 12a and 12b are attached to several spots on the abdominal wall. A laparoscope 2 (hereinafter also referred to as an endoscope), an energy treatment tool 3, forceps 4, and the like are inserted into the body through holes provided in the trocars 12a and 12b. Then, a procedure such as excision of an affected site 16 (tumor or the like) using the energy treatment tool 3, etc. is performed while watching in real time an image of the affected site 16 video-imaged by the endoscope 2. The endoscope 2, the energy treatment tool 3, and the forceps 4 are held by an operator, an assistant, a scopist, a robot, or the like.

In the operating room where such endoscopic surgery is performed, a cart 14 on which apparatuses for the endoscopic surgery are mounted, a patient bed 13 on which the patient is lying, a foot switch 15, and the like are disposed. As medical devices, for example, apparatuses such as a camera control unit (CCU) 5, a light source apparatus 6, a treatment tool apparatus 7, an insufflator 8, a display 9, a recorder 10, a printer 11, etc. are placed on the cart 14.

An image signal of the affected site 16 captured through an observation optical system of the endoscope 2 is transmitted to the CCU 5 via a camera cable. After being subjected to signal processing in the CCU 5, the image signal is outputted to the display apparatus 9, and an endoscopic image of the affected site 16 is displayed. The CCU 5 may be connected to the endoscope 2 via the camera cable, or may be wirelessly connected to the endoscope 2.

The light source apparatus 6 is connected to the endoscope 2 via a light guide cable, and is able to irradiate the affected site 16 with light of various wavelengths switchably. The treatment tool apparatus 7 is, for example, a high frequency output apparatus that outputs a high frequency current to the energy treatment tool apparatus 3 that uses electric heat to cut the affected site 16.

The insufflator 8 includes air-supply and air-suction means and supplies air into the body of the patient such as an abdominal region. The foot switch 15 controls the CCU 5, the treatment tool apparatus 7 and the like with foot manipulation by the operator, the assistant, etc., as a trigger signal.

### 2. Overview of Surgical System According to Present Disclosure

In recent years, an operating room has been a system in which devices are connected by an IP network. The present disclosure provides a system in which a server changes a control parameter of a selected device to achieve image quality desired by a user in a surgical system connected by the IP network.

In addition, in this system, the server grasps a controllable and adjustable device in the surgical system and, if there is a plurality of such devices, the server changes the control parameter of one, among those devices, that is most suitable for an intended use. Moreover, in this system, the server decides on selection of those devices by evaluating necessary conditions, effects of adjustments or ranges of influences, etc.

In addition, in this system, connection by the IP network to devices to be connected may be established directly or via an IP converter (IPC). Targets to be controlled by the server include the IP converter.

Furthermore, in addition to image quality adjustment, the surgical system performs adjustment of synchronization (latency) of images in, e.g., P in P, sound quality adjustment, adjustment of synchronization (latency) of sound with images, and adjustment of hue among a plurality of monitors. In addition, when adjustment is performed by the server, the server outputs a test signal for adjustment, and performs coordination of the devices such powering-off of a device that interferes with the adjustment, and the like.

### 3. Configuration Example of Surgical System

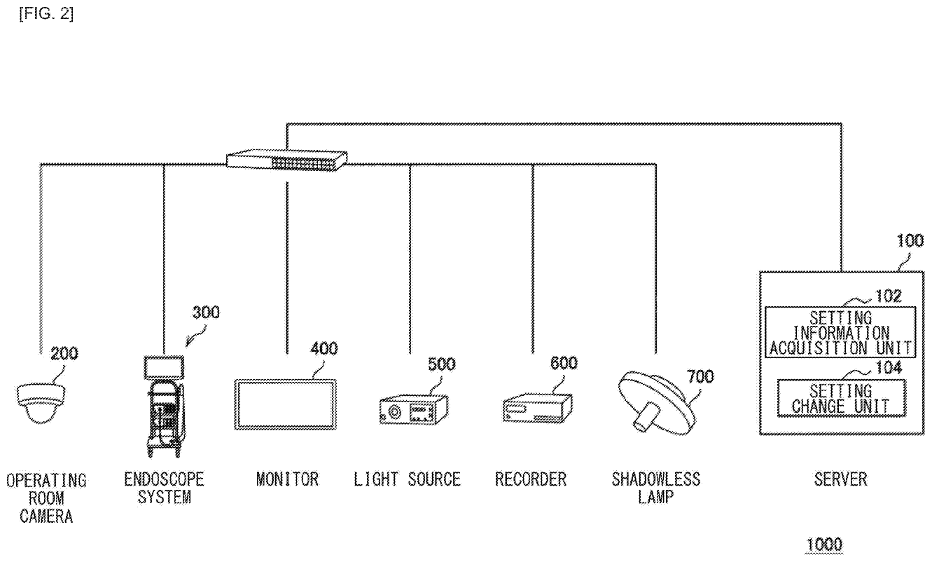

FIG. 2 is a schematic diagram illustrating a surgical system 1000 connected to the IP network. The surgical system 1000 includes a server 100, an operating room camera 200, an endoscope system 300, a monitor 400, a light source 500, a recorder 600, and a shadowless lamp 700. In the surgical system 1000 illustrated in FIG. 2, images, control commands, statuses, etc. are transmitted and received over the IP network.

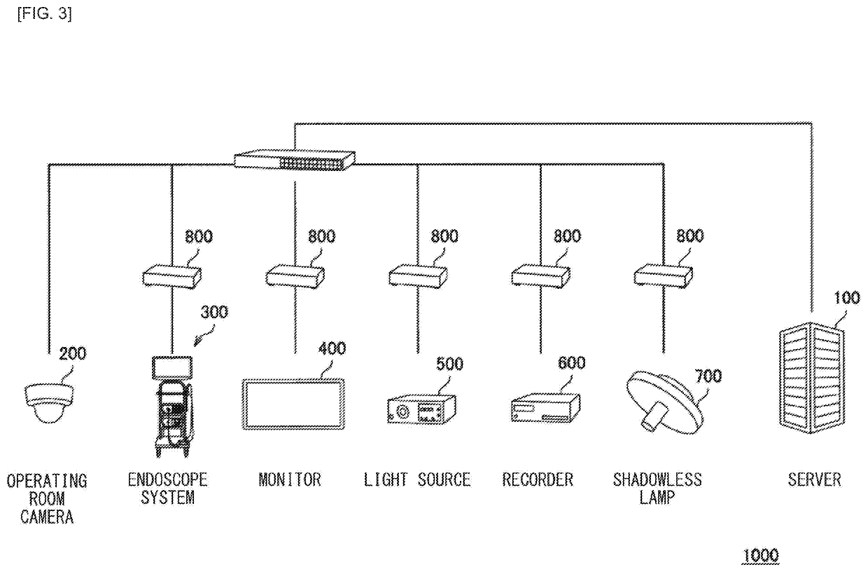

FIG. 3 is a schematic diagram illustrating a configuration example in which in a case where each of the devices illustrated in FIG. 2 does not have an interface of the IP network, the IP converter 800 performs interface conversion between that device and the IP network. The IP converter 800 also has a function of performing image processing or the like, in addition to the interface conversion. The server 100 illustrated in FIG. 2 and FIG. 3 is able to control, via the IP network, a device connected directly, a device connected via the IP converter 800, or the IP converter 800 itself.

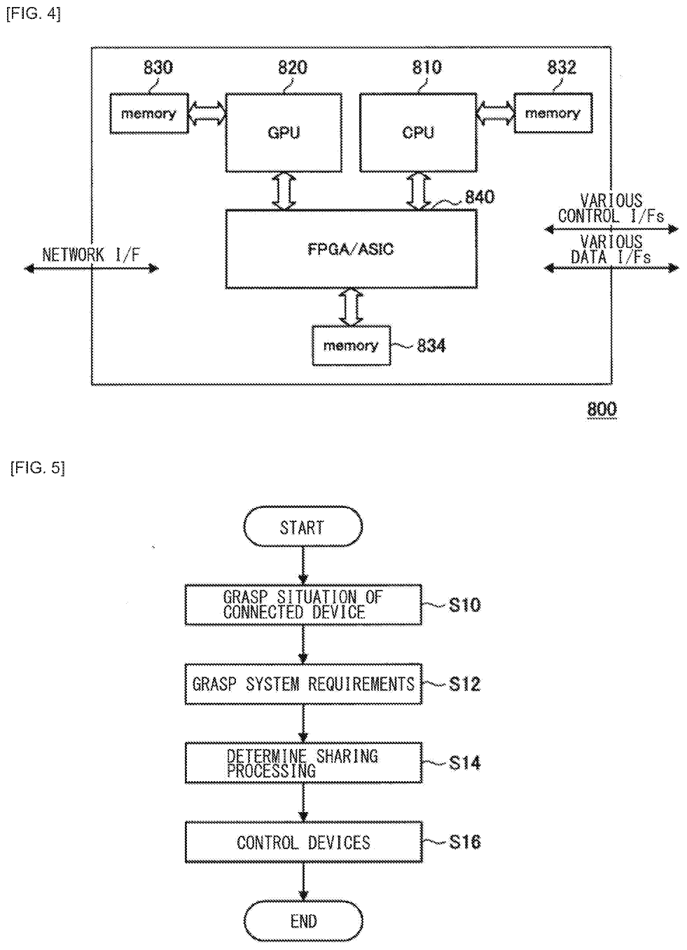

FIG. 4 is a schematic diagram illustrating a configuration of the IP converter 800. As illustrated in FIG. 4, the IP converter 800 includes a CPU 810, a GPU 820, memories 830, 832, and 834, and an FPGA/ASIC 840.

In addition, the IP converter 800 includes a network I/F for connection to the server 100, etc., and various types of data I/Fs and control I/Fs for connection of the devices. In a case where a device to be connected is a camera or the like, the IP converter 800 transmits data from the device to the server 100. In addition, in a case where the device to be connected is the recorder 600 or the like, the IP converter 800 outputs data from the server 100 to each of the devices. At this time, the IP converter 800 transfers a control command from the server to the device with a protocol changed on the basis of the type of the device, and provides output to the device. For example, in a case where the IP converter 800 recognizes that the connected device is the camera or the recorder 600, the IP converter 800 transfers the control command from the server 100 to the device with the protocol changed, and provides output to the device.

It is to be noted that as data transfer, inter-device transfer may be performed through the IP converters 800 without going through the server 100.

According to the present disclosure, in the surgical system connected via the IP network, the server 100 grasps a situation of each of the devices in the system in relation to an intended adjustment, determines sharing of the adjustment among the devices in accordance with system requirements, and performs the adjustment.

As illustrated in FIG. 2, the server 100 includes a setting information acquisition unit 102 and a setting change unit 104. The setting information acquisition unit 102 acquires information to calibrate each of the devices such as the monitor 400. The setting change unit 104 changes settings of each of the devices such as the monitor 400 on the basis of the information acquired by the setting information acquisition unit 102. The server 100 also includes a database (not illustrated) that retains various types of information. The database may be provided separately from the server 100. Additionally, it is possible for these components of the server 100 to include hardware such as a circuit, a central processing unit such as a CPU, and a program for causing the CPU to function. The server 100 illustrated in FIG. 3 is configured similarly to FIG. 2.

The server 100 acquires two or more pieces of information related to the plurality of devices connected via the IP network and changes at least one of settings of the plurality of devices on the basis of the two or more pieces of information. The information related to the plurality of devices includes information acquired from output signals from the devices and, in a case where one of the devices is the monitor, image information acquired by capturing of an image of the monitor by another device. In addition, the information related to the plurality of devices also include information regarding a test pattern of an image or the like. It is possible for the server 100 to evaluate an adjustable range, the adjustment effect, the range of influence, and the like of each of the devices and to determine a device to be adjusted on the basis thereof. For example, it is possible for the server 100 to determine, with a device having a narrower adjustable range as a reference, a device having a wider adjustable range to be a device targeted for adjustments.

FIG. 5 is a flowchart illustrating basic processing of the system. First, in a step S10, the server 100 grasps connected device information. In a next step S12, the server 100 grasps the system requirements. In a next step S14, the server 100 determines sharing processing. In a next step S16, the server 100 controls the devices.

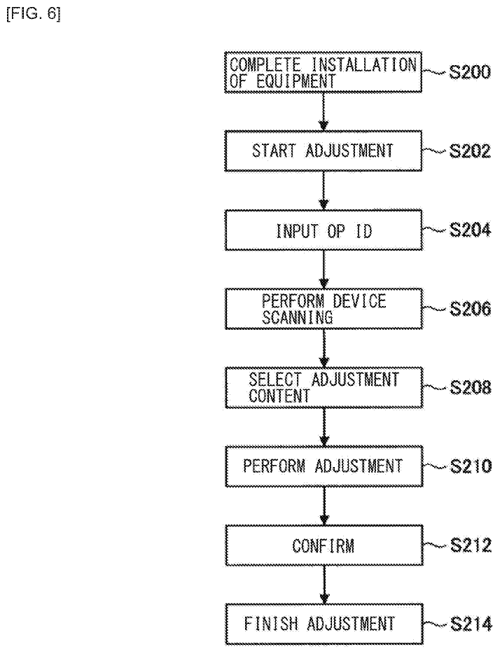

In addition, FIG. 6 is a flowchart that illustrates FIG. 5 in more detail and illustrates processing from installation of each of the devices of the surgical system 1000 to the end of the adjustment of the device. A device coordinator of the operating room performs adjustment work when the devices to be used are ready in the operating room. Although the adjustment work is executed by operation and input from a user with a tablet terminal 850 connected to the IP network or a terminal similar thereto, actual adjustment control is performed by the server 100.

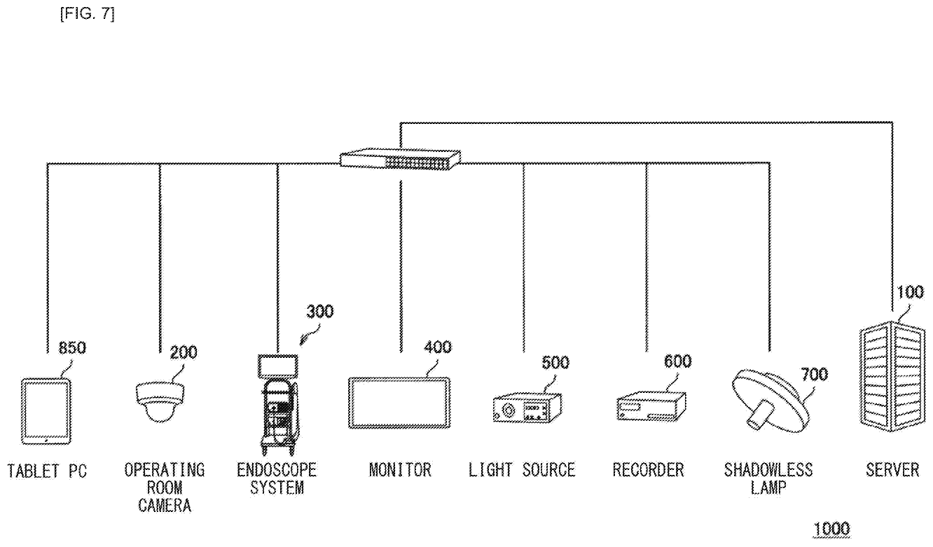

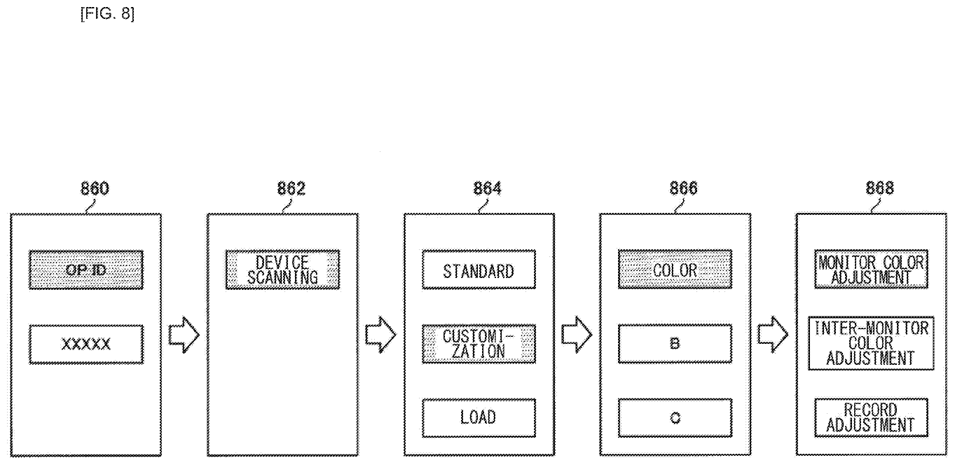

Regarding processing illustrated in FIG. 6, it is possible for the user operating the tablet terminal to perform the processing of step S204 to step S212. FIG. 7 is a schematic diagram illustrating the surgical system 1000 in which the tablet terminal is added to the surgical system of FIG. 2. It is possible to configure the tablet terminal 850 to wirelessly communicate with the server 100. In addition, FIG. 8 is a schematic diagram illustrating a transition on a display screen of the tablet terminal 850.

In FIG. 6, first, in a step S200, installation of each of the devices in the operating room and other necessary rooms is completed. In a next step S202, adjustment of each of the devices is started. In a next step S204, the user inputs an operation ID (OP ID).

The operation ID inputted is transmitted to the server 100. FIG. 8 illustrates an input screen 860 of the operation ID in the step S204.

In a case where information regarding an operative procedure, an operator, nurses, etc. are registered in the operation ID, it is possible to load, from the database, standard equipment information and an adjustment menu of that operative procedure, preference data that matches the operative procedure associated with the operator, individual-specific data associated with the operator or the like, etc., and to reflect them in the adjustment contents for the operation.

Thus, in a case where surgery information is managed as the operation ID on the server 100, the coordinator inputs the operation ID. It is assumed that information regarding device settings such as the operative procedure, the operator, the nurses, etc. are registered in the operation ID. Reference to the operation ID makes it possible to load, from the database of the server 100, the standard equipment information and the settings thereof based on operative procedure information, equipment settings that are preferred by the operator based on operator information, individual-specific data such as hearing ability based on information regarding the operator and the nurses, etc., and to reflect the loaded information in the adjustments.

In a next step S206, the server 100 performs scanning of each of the devices. Here, the server 100 confirms the surgical devices connected via the IP network. This allows the server 100 to acquire detailed information regarding each of the devices. The server 100 also makes inquiries to each of the devices about an adjustable parameter by way of IP communications over the IP network. In addition, the server 100 grasps adjustment contents stored in the database. FIG. 8 illustrates an input screen 862 of the device scanning in the step S206.

The device coordinator performs retrieval of the IP network and a controllable device in the operating room by selecting and executing the device scanning on the tablet terminal 850. A mechanism such as a bridge makes it possible for the tablet terminal 850 to identify that the tablet terminal 850 is located in the operating room. It is possible for the server 100 to identify a connected device from the operation ID or the like acquired through the device scanning and to grasp whether or not the device has an adjustment mechanism, the range of adjustment for the device, etc. The information may be grasped from a response to the inquiry communication from the server 100 or on the basis of the information regarding the devices registered on the database.

In a next step S208, adjustment contents are selected. By way of example, the adjustment contents are selectable from “standard”, “customization”, and “load (calling registered data and new adjustment)”. Furthermore, the adjustment contents are selectable from “color”, “sound”, and “amount of delay”. FIG. 8 illustrates an input screen 864, an input screen 866, and an input screen 868 for selection of the adjustment contents in the step S208. When “customization” is selected on the input screen 864, the input screen 866 is displayed. In addition, when “color” is selected on the input screen 866, the input screen 868 is displayed. It is possible for the user to select monitor color adjustment, inter-monitor color adjustment, or record adjustment (adjustment of a record by the recorder 600) on the input screen 868. The input screen 868 indicates that the monitor color adjustment has been selected.

In this manner, functions adjustable in the operating room are listed and displayed on the tablet terminal 850. On the tablet terminal 850, it is possible to select standard setting or new customization, loading of setting information or the like, and, in a case where customization is selected, various functions are displayed.

In a next step S210, various adjustments are performed. Note that specific examples of the various adjustments will be described later. In a next step S212, the adjustment contents are confirmed. If necessary, fine adjustment is performed manually, and whether or not to save adjustment data is selected. In a next step S214, the adjustment is finished.

The coordinator selects and executes a necessary function from the menu. After executing, the coordinator checks the adjustment contents. It is possible for the coordinator to give an instruction, from the tablet terminal 850, to change a method of adjustment in a case where the adjustment not intended by the coordinator has been performed, and to perform fine adjustment of an adjusted value in a case where the fine adjustment is necessary. When the adjustment is complete, it is possible to save the adjustment data for use as next adjustment data. The adjustment data is stored in and managed by the database on the server 100. The standard setting is setting data that is stored as one defined by hospital facilities. In addition to this, it is possible to specify and load data stored at the time of the adjustment. Moreover, in a case where a fine adjustment is done at the operator's desire after adjustment by the coordinator, saving a result thereof makes it possible to provide the operator's intended settings in next adjustments. Even in a case where all the adjustments are ones manually done by the coordinator, it is possible to store and manage them in a similar manner.

Although manual work by the coordinator such as changing a camera position may occur depending on the adjustment contents, all necessary work is instructed on the tablet terminal 850 and the work is done in accordance with the instructions. This makes it possible for anyone to equally perform adjustments. In addition, work that involves timing synchronization among devices is controllable from the server 100, which makes it easy to perform the work. In the following, description is given of specific adjustments of the devices.

### 4. First Embodiment

In a first embodiment, description is given of adjustment of a color of the monitor 400, adjustment of the endoscope system 300, adjustment of the light source 500, etc. Examples of the purposes thereof include correctly outputting a color of the inside of the abdominal cavity to the monitor 400 during scopic surgery or the like. Here, description is also given of a case of using a calibration camera 900, characteristics of which have been grasped.

### 4.1. Adjustment of Monitor

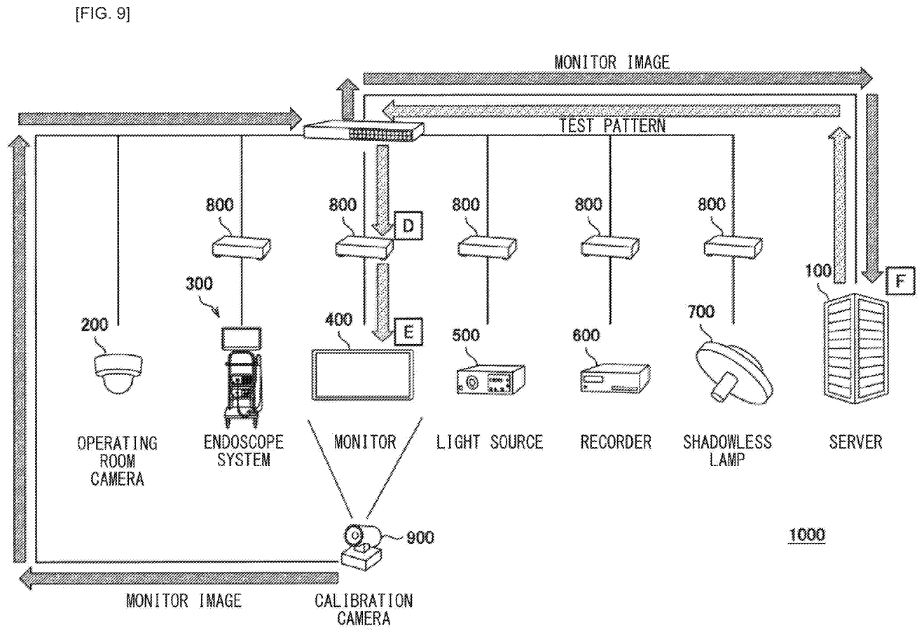

FIG. 9 is a schematic diagram illustrating how the monitor 400 is adjusted in the configuration of the surgical system 1000 illustrated in FIG. 3. As illustrated in FIG. 9, a test pattern for display is transmitted from the server 100 to the monitor 400, and the test pattern is displayed on the screen of the monitor 400. Then, the monitor 400 is imaged by the calibration camera 900. A monitor image acquired by the imaging is transmitted to the server 100. The server 100 compares the test pattern and the monitor image captured by the camera 900, and performs calibration of the monitor 400 on the basis of a result of the comparison. The target of the adjustment by the server 100 may be the monitor 400 itself or the IP converter 800 provided for the monitor 400. Therefore, D and E illustrated in FIG. 9 are adjustment spots.

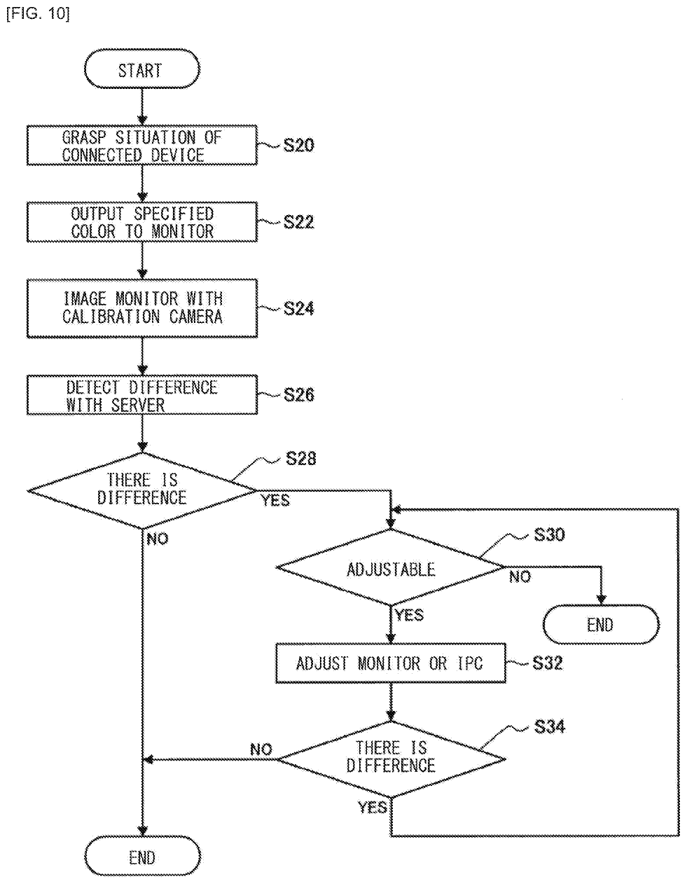

FIG. 10 is a flowchart illustrating processing related to the adjustment of the monitor 400. First, in a step S20, the server 100 grasps a situation of the connected device (the monitor 400 or the IP converter 800 of the monitor) in a system to the output of the monitor, and acquires detailed information regarding each of the devices. In a next step S22, the server 100 causes the monitor 400 to display a test pattern of a specified color. It is to be noted that although data of the test pattern is generated by the server 100 by way of example, the data may be generated by the monitor 400 or the IP converter 800 of the monitor. In a next step S24, the test pattern displayed on the screen of the monitor 400 is imaged by the calibration camera 900. In a next step S26, the server 100 acquires a difference between the test pattern that the server 100 has caused the monitor 400 to display and the test pattern on the screen of the monitor 400 imaged by the camera 900.

In a next step S28, it is determined whether or not there is a difference between the test pattern that the monitor 400 has been caused to display and the test pattern on the screen of the monitor 400 imaged by the camera 900. In a case where there is a difference, processing proceeds to a step S30. In addition, in a case where there is no difference in a step S28, the processing ends.

In the step S30, it is determined whether or not the difference is adjustable. In a case where the difference is adjustable, the processing proceeds to a step S32. In the step S32, adjustment is performed to minimize the difference between the test pattern displayed on the monitor 400 and the test pattern retained on the server 100 side by adjusting the monitor 400 or the IP converter 800 connected with the monitor 400.

Following the step S32, the processing proceeds to a step S34. In the step S34, it is determined whether or not there is a difference between the test pattern that the monitor 400 is caused to display and the test pattern on the screen of the monitor 400 imaged by the camera 900. In a case where there is a difference, the processing returns to the step S30 and the subsequent processing is performed again. In contrast, in a case where there is no difference in the step S34, the processing for adjustment of the monitor 400 ends. The above processing makes it possible to match a display state of the monitor 400 with the test pattern.

### 4.2. Adjustment of Endoscopic System

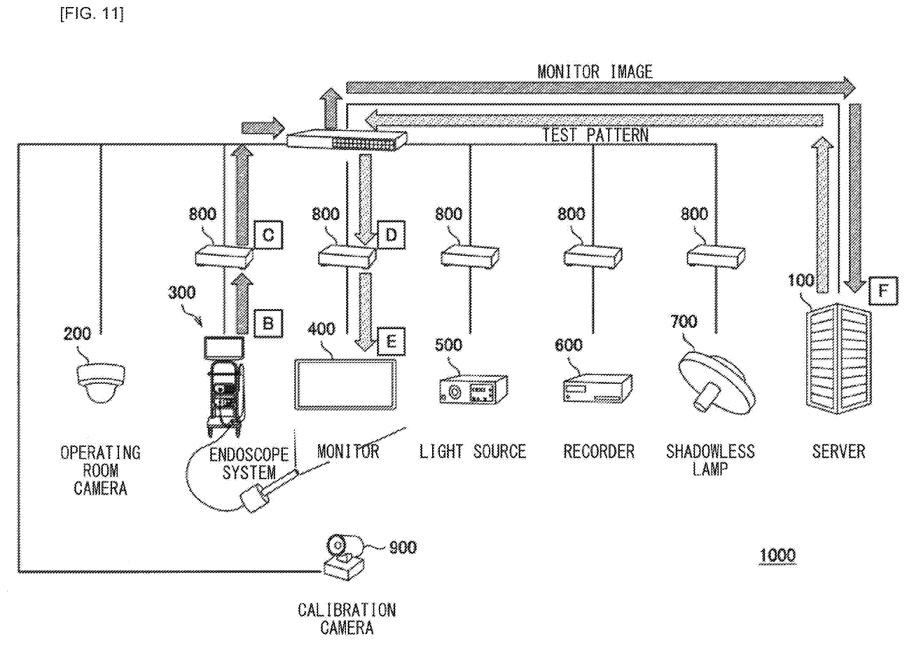

FIG. 11 is a schematic diagram illustrating how the endoscope system 300 is adjusted in the configuration of the surgical system 1000 illustrated in FIG. 3. The endoscope system 300 includes an endoscope to be inserted into the body. The endoscope includes a camera (endoscope camera). The endoscope system 300 also includes a camera control unit (CCU) that controls the endoscope camera.

As illustrated in FIG. 11, a test pattern for display is transmitted from the server 100 to the monitor 400, and the test pattern is displayed on the screen of the monitor 400. Then, the monitor 400 is imaged by the endoscope system 300. The monitor image acquired by the imaging is transmitted to the server 100. The server 100 compares the test pattern and the monitor image captured by the endoscope system 300, and performs the calibration of the endoscope system 300 on the basis of a result of the comparison. The target of the adjustment by the server 100 may be the endoscope system 300 itself or the IP converter 800 provided for the endoscope system 300. Therefore, B and C illustrated in FIG. 11 are the adjustment spots.

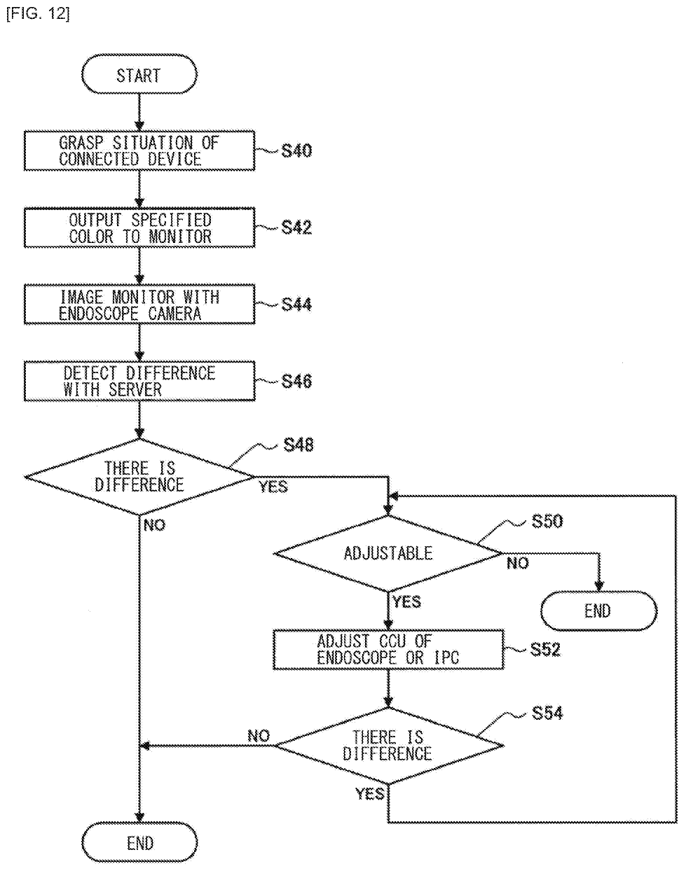

FIG. 12 is a flowchart illustrating processing related to the adjustment of the endoscope system 300. First, in a step S40, the server 100 grasps the situation of the connected device and acquires detailed information regarding each of the devices. In a next step S42, the server 100 causes the monitor 400 to display the test pattern of a specified color. It is assumed here that the monitor 400 has already been subjected to the calibration that is described in the first embodiment. It is to be noted that although the data of the test pattern is generated by the server 100 by way of example, the data may be generated by the monitor 400 or the IP converter 800 of the monitor. In a next step S44, the test pattern displayed on the screen of the monitor 400 is imaged by the camera of the endoscope system 300. In a next step S46, the server 100 acquires a difference between the test pattern that the serve 100 has caused the monitor 400 to display and the test pattern on the screen of the monitor 400 imaged by the camera of the endoscope system 300. It is to be noted that the server 100 acquires image data of the test pattern that has been processed by the camera control unit of the endoscope system 300 and acquires a difference from the test pattern that the monitor 400 has been caused to display.

In a next step S48, it is determined whether or not there is a difference between the test pattern that the monitor 400 has been caused to display and the test pattern on the screen of the monitor 400 imaged by the camera of the endoscope system 300. In a case where there is a difference, the processing proceeds to a step S50. In addition, in a case where there is no difference in the step S48, the processing ends.

In the step S50, it is determined whether or not the difference is adjustable. In a case where the difference is adjustable, the processing proceeds to a step S52. In the step S52, adjustment is performed to minimize the difference between the test pattern displayed on the monitor 400 and the test pattern imaged by the camera of the endoscope system 300 by adjusting the camera control unit of the endoscope system 300 or the IP converter 800 connected with the endoscope system 300.

Following the step S52, the processing proceeds to a step S54. In the step S54, it is determined whether or not there is a difference between the test pattern that the monitor 400 is caused to display and the test pattern imaged by the camera of the endoscope system 300. In a case where there is a difference, the processing returns to the step S50, and the subsequent processing is performed again. In contrast, in a case where there is no difference in the step S54, the processing for adjustment of the endoscope system 300 ends. The above processing makes it possible to match the image captured by the endoscope camera with the test pattern.

### 4.3. Adjustment of Light Source

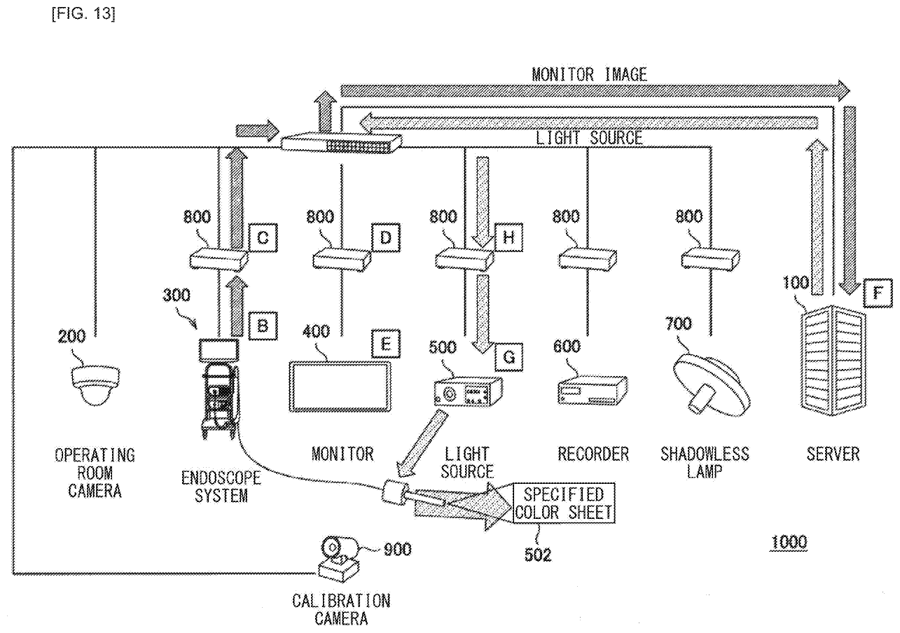

FIG. 13 is a schematic diagram illustrating how the light source 500 is adjusted in the configuration of the surgical system 1000 illustrated in FIG. 3. As illustrated in FIG. 13, the server 100 transmits to the light source 500 a command to project predetermined light, and the light source 500 projects light to a sheet 502 of a specified color (for example, white). The light projected from the light source 500 is applied to the sheet 502 via the endoscope of the endoscope system 300. Then, the sheet 502 is imaged by the endoscope camera having undergone calibration. An image of the sheet 502 acquired by the imaging is transmitted to the server 100. The server 100 compares a color that the server 100 has specified for the light source 500 to project and a color of the sheet 502 imaged by the endoscope camera, and performs calibration of the light source 500 on the basis of a result of the comparison. The target of the adjustment by the server 100 may be the light source 500 itself or the IP converter 800 provided for the light source 500. Therefore, G and H illustrated in FIG. 13 are the adjustment spots.

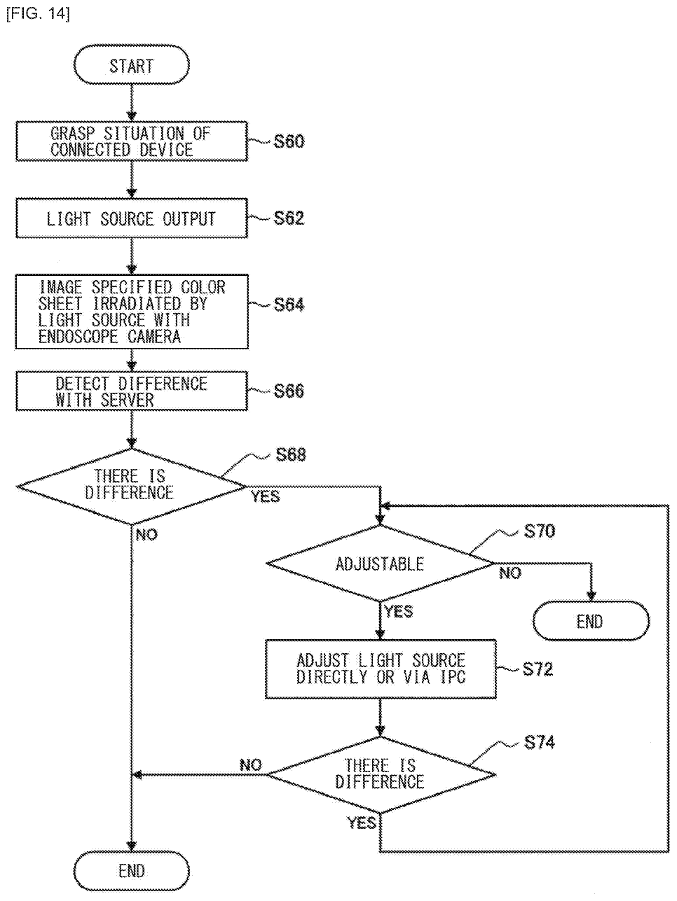

FIG. 14 is a flowchart illustrating processing related to the adjustment of the light source 500. First, in a step S60, the server 100 grasps the situation of the connected device and acquires detailed information regarding each of the devices. In a next step S62, the server 100 causes the light source 500 to irradiate the sheet 502 with predetermined light. In a next step S64, the sheet 502 irradiated with the light from the light source 500 is imaged by the endoscope camera. In a next step S66, the server 100 acquires a color difference between the light applied from the light source 500 and the image of the sheet 502 captured by the endoscope camera.

In a next step S68, it is determined whether or not there is a color difference between the light projected from the light source 500 and the image of the sheet 502 captured by the endoscope camera. In a case where there is a difference, the processing proceeds to a step S70. In addition, in a case where there is no difference in the step S68, the processing ends. It is to be noted that a determination on whether or not there is a difference is made in consideration of an influence of the color of the sheet 502 on the image captured by the endoscope camera.

In the step S70, it is determined whether or not the difference is adjustable. In a case where the difference is adjustable, the processing proceeds to a step S72. In the step S72, adjustment is performed to minimize the color difference between the light projected from the light source 500 and light that is assumed on the server 100 side by adjusting the light source 500 or the IP converter 800 connected with the light source 500.

Following the step S72, the processing proceeds to a step S74. In the step S74, it is determined whether or not there is a color difference between the light projected from the light source 500 and the image of the sheet 502 captured by the endoscope camera. In a case where there is a difference, the processing returns to the step S70 and the subsequent processing is performed again. In contrast, in a case where there is no difference in the step S74, the processing for adjustment of the light source 500 ends. The above processing makes it possible to adjust the light projected from the light source 500 into a desired state.

### 4.4. In a Case Where There Remains Error in Each Adjustment

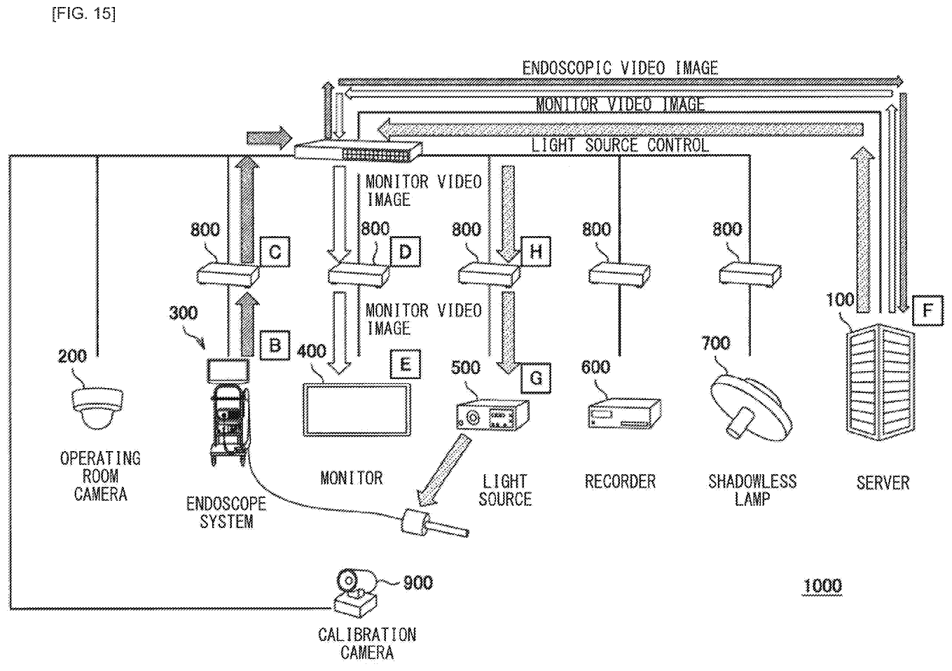

Description is given of processing in a case where an error remains in the adjustment of the monitor 400, the adjustment of the endoscope system 300, and the adjustment of the light source 500 described above. FIG. 15 is a diagram illustrating the processing for system adjustment in the case where an error remains in the adjustment of the monitor 400, the adjustment of the endoscope system 300, and the adjustment of the light source 500. In a case where there remains an adjustment error, the server 100 performs adjustment to eliminate the error by adjusting the monitor 400 or the IPC converter 800 of the monitor 400, the endoscope system 300 or the IPC converter 800 of the endoscope system 300, and the light source 500 or the IP converter 800 of the light source 500 in an integral manner. In addition, in a case where the system requirements are satisfied even via the server 100, the adjustment may be performed by processing on the server 100. Therefore, B, C, D, E, F, G, and H illustrated in FIG. 13 are the adjustment spots.

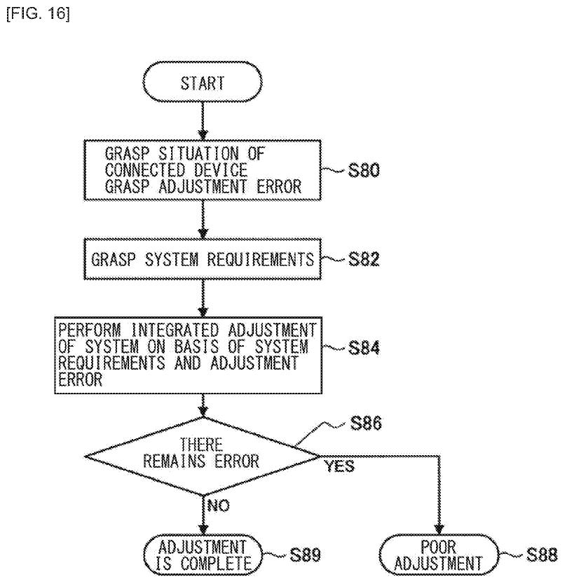

FIG. 16 is a flowchart illustrating the processing for system adjustment in a case where an error remains. First, in a step S80, the server 100 grasps the situation of the connected devices. The server 100 acquires detailed information regarding each of the devices and grasps the adjustment error. In a next step S82, the server 100 grasps the system requirements. Here, the system requirements refer to, for example, a requirement that a latency (delay) up to when an image captured by the endoscope camera of the endoscope system 300 is outputted to the monitor 400 be within one frame, and the like.

In a next step S84, an integrated adjustment of the system is performed on the basis of the system requirements and the adjustment error. By way of example, in the case where the latency is the system requirement as described above, the server 100 determines an amount of adjustment and performs the adjustment within the range of the system requirement in consideration of a characteristic and an amount of adjustment range of each of the devices to allow a subject photographed by the endoscope camera of the endoscope system 300 to be correctly displayed on the monitor 400.

In a next step S86, it is determined whether or not as a result of the integrated adjustment performed in the step S84, there remains the adjustment error (difference). In a case where there remains the difference, the processing proceeds to a step S88 where it is determined that the adjustment is poor. In contrast, in a case where there remains no error in the step S86, the processing proceeds to a step S89 where it is determined that the adjustment is complete.

It is to be noted that in a case where photographing is performed with the calibration camera 900 or the camera of the endoscope system 300, the server 100 controls timing to turn off or to photograph in order to eliminate an influence of illumination in the operating room or the shadowless lamp.

### 5. Second Embodiment

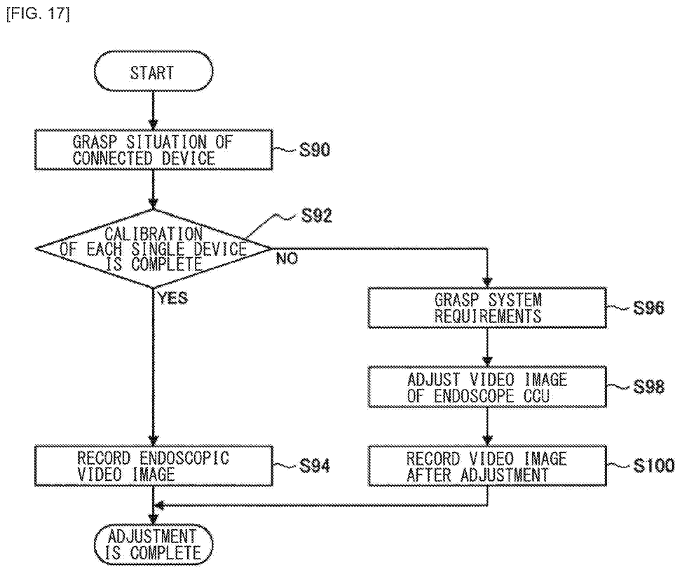

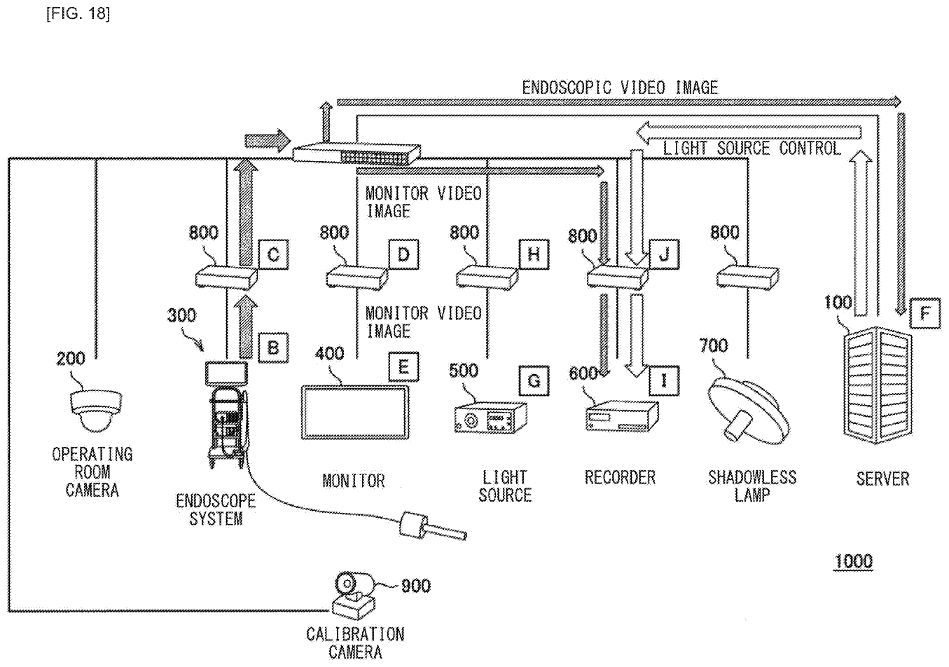

In a second embodiment, adjustment of the recorder 600 (video image recorder) is performed in the first embodiment. The recorder 600 mainly records a video image of the endoscope camera of the endoscope system 300. Examples of the purposes thereof include correctly recording the color of the inside of the abdominal cavity. FIG. 17 is a flowchart illustrating processing related to the second embodiment. First, in a step S90, the server 100 grasps the situation of the connected device and acquires detailed information regarding each of the devices. In a next step S92, it is determined whether or not calibration of each single device is complete. In a case where the calibration is complete, the processing proceeds to a step S94. It is to be noted that the calibration of each single device includes the calibration described in the first embodiment. In the step S94, the recorder 600 records the video image of the endoscope camera.

In addition, in a case where the calibration of each single device is not complete in the step S92, the processing proceeds to a step S96. In this case, each single device is unadjusted but the devices have been integrally adjusted. In the step S96, the server 100 grasps the system requirements. An example of the system requirements may be the above-described example of the latency.

In the case of video image recording, it is not necessary that the timing of video image acquisition and the timing of video image recording perfectly coincide with each other, and therefore any latency from the endoscope camera of the endoscope system 300 to the recorder 600 presents no problem. In a step S98, an adjustment spot that meets the system requirement is extracted and adjusted. In addition to adjustments performed at the endoscope system 300 and the recorder 600, it is also possible to perform latency adjustments on the server 100 side by allowing the server 100 to adjust the latency of the video image acquired by the endoscope system 300 and send the adjusted video image to the recorder 600. FIG. 18 is a schematic diagram illustrating adjustment spots. In this case, F (server 100), I (recorder 600), or J (IP converter 800) is an adjustment spot. Thus, processing on the server 100 is also a target of adjustment.

For the step S98, assume that the server 100 has selected the endoscope system 300 as the adjustment spot. Thus, in the step S98, the latency of the video image of the CCU of the endoscope system 300 is adjusted. As described above, it is possible for the server 100 to determine on selection of the adjustment spot. In a next step S100, the video image after the adjustment is recorded by the recorder 600.

### 6. Third Embodiment

In a third embodiment, adjustments are performed to suit the preference of the user of the surgical system 1000 such as the operator. Examples of the purposes thereof include performing appropriate adjustments of the monitor color for the operator when performing scopic surgery. In addition to adjustment of the monitor color in a case where different operators have different color preferences for the monitor 400, adjustment of a difference in hue resulting from different manufacturers of the monitor 400, for example, is also possible.

It is possible to accumulate, in the database included in the server 100 or the database connected to the server 100, information indicating what settings the operator selected for what operative procedure with what kind of equipment. The database is accessible from inside or outside the operating room, or regardless of whether inside or outside the hospital.

First, the server 100 analyzes the environment preference of the operator from the operator information. As a precondition, the operator enters information such as his or her own ID into the server 100 from the tablet terminal 850. The server 100 extracts, from the database, surgery information of the operator registered in advance. The server 100 determines the operator's preference applied in a case where the operator performed a similar operation on a similar site, and determines an environment that seems to be optimal for the operator.

Because the server 100 grasps the information regarding the connected devices, the server 100 calculates parameters of the devices that fit the operator's preference on the basis of the situation of the equipment in the operating room at that time. For example, if the operator has a preferred monitor color, the server 100 calculates an appropriate parameter for the monitor color. In addition, in a case where there is equipment that is not controllable, the server 100 performs integrated adjustment as the system. For example, in a case where the color is not adjustable on the monitor 400 side, an approach is taken in which the endoscope system 300 side is substituted for the unadjustable equipment to perform the adjustment, or the like.

### 7. Fourth Embodiment

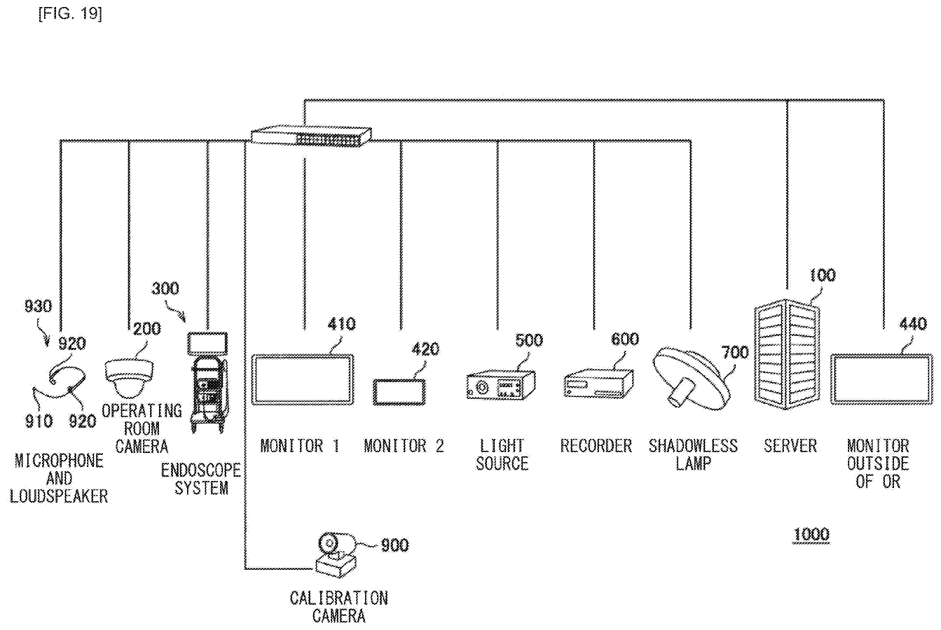

In a fourth embodiment, in a case where there are two monitors 400 in the operating room, hue adjustment for the two monitors 400 is performed. FIG. 19 is a schematic diagram illustrating an extended surgical system 1000. In the system illustrated in FIG. 19, two monitors 410 and 420 are provided. Differences between the two monitors 410 and 420 in setting, manufacturer, etc. may result in different hues or different image appearances. In such a case, it is possible to adjust the hue or image appearance by adjusting either one of the monitors.

First, the server 100 grasps information regarding the connected device with the calibration camera 900 ready. At this time, the server 100 grasps the situations of the monitors 410 and 420 and acquires detailed information regarding each of the devices. Examples of the situations of the monitors 410 and 420 include whether or not adjustment is possible, a magnitude of an amount of adjustment, etc. On the basis of a result of grasping the information regarding the monitors 410 and 420, the server 100 assumes one of the two monitors 410 and 420 that is not adjustable or is smaller in the amount of adjustment as a reference monitor.

Then, the server 100 instructs the two monitors 410 and 420 to output the same video image data. Alternatively, the server 100 transmits the same video image data to the two monitors 410 and 420. After that, the calibration camera 900 photographs the two monitors 410 and 420. The photographed images of the two monitors 410 and 420 are transmitted to the server 100. The server 100 calculates a difference between the photographed images of the two monitors 410 and 420, and adjusts one of the monitors that is not the reference monitor.

### 8. Fifth Embodiment

In a fifth embodiment, the endoscope image and voice data are recorded in synchronization. The surgical system 1000 illustrated in FIG. 19 includes a microphone 910 and a loudspeaker 920. The microphone 910 and the loudspeaker 920 may be provided on a head set 930 or may be provided separately. Provision of the microphone 910 and the loudspeaker 920 on the head set 930 allows the users in the operating room or outside of the operating room to have a conversation with each other by wearing the head set 930.

The server 100 outputs, from the loudspeaker 920, voice in the operating room acquired from the microphone 910. The server 100 also outputs images acquired from the endoscope camera of the endoscope system 300, the operating room camera 200 and the like to the monitor 410 (or the monitor 420) in the operating room and a monitor 440 provided outside of the OR. At this time, if the video image displayed on the monitor 440 and the voice outputted from the loudspeaker 920 are not in synchronization, the user viewing the video image using the monitor 440 outside of the operating room feels strange.

To avoid this, processing to synchronize the video image displayed on the monitor 440 with the voice outputted from the loudspeaker 920 is performed on the server 100 side. In the synchronization adjustment at the server 100, processing is performed to provide an appropriate delay.

The server 100 grasps the situation of each of the devices in the system and acquires detailed information regarding each of the devices. It therefore is possible to grasp in advance an amount of delay between the devices. Then, in a case where the amount of delay between the voice and the video image is clear, the server 100 adjusts a path with a smaller amount of delay to match a path with a larger amount of delay. For example, the server 100 acquires, as known information from the microphone 910 and the loudspeaker 920, the amount of delay from when voice information acquired by the microphone 910 is transmitted to the server 100 to when the voice information is transmitted from the server 100 to the loudspeaker 920 and the voice is outputted from the loudspeaker 920. The server 100 also acquires the amount of delay from when the image captured by the endoscope camera is transmitted to the server 100 to when the image information is transmitted from the server 100 to the monitor 400 and the image is outputted from the monitor 400. The server 100 adjusts timing of outputting the voice information to the loudspeaker 920 or timing of outputting the image information to the monitor 400 to match these amounts of delay acquired.

It is to be noted that because the video image to be viewed by the operator in the operating room preferably has no delay, processing of synchronizing the video image with the voice outputted from the loudspeaker 920 is not necessary. That is, it is preferable to perform the processing of synchronizing only the video image outputted to the outside of the operating room with the voice outputted from the loudspeaker 920.

For example, in a case where the video image is processed by any one of the devices and thereby delayed by one frame from the voice, the server 100 performs adjustment to also delay the voice by one frame to allow both the video image and the voice to be simultaneously inputted to the recorder 600. In a case where the voice is recorded along with time information, the server 100 performs, e.g., shifting of the time information to prevent the time information, the video image, and the voice from being out of synchronization.

In contrast, in a case where the amount of delay between the voice and the video image is unknown, a delay measurement mode is set. For example, the user's hands are imaged by the endoscope camera, and the hands are recognized by image processing. While confirming that the recognized hands are displayed on the monitor 400, an input of an image and sound corresponding to each other is made to the surgical system 1000 by, for example, clapping the hands within the screen of the monitor 400. The server 100 acquires the sound from the microphone 910 and the image from the endoscope camera, and checks a pulsed waveform of the sound at the time of the clapping of the hands against the image at the timing when the hands are clapped that is acquired by image processing. Thus, the server 100 grasps the amount of delay between the image and the sound from their respective timings. If loudness of the sound is outputted on the monitor 400 with something like a volume bar, it becomes possible to inform the user that detection of the action of clapping the hands has been successful and that the sound has been a detectable one. Then, the server 100 adjusts a path with a smaller amount of delay to match a path with a larger length of delay.

### 9. Sixth Embodiment

In a sixth embodiment, adjustments of voice volume and voice quality are performed. Examples of the purposes thereof include performing adjustments of the volume and quality of voice recorded by the recorder 600, and optimizing the volume of voice during conversations using the head set 930. When sound is recorded, it is desirable that voice from the operator, etc. be recorded as clearly as possible. In the operating room, various devices produce sound and thus there may be a case in which an instruction from the operator fails to reach a nurse at a position away from the operator. Also, there may be a case in which the voice of the nurse fails to reach the operator. Moreover, there may be a case in which communication with outside of the operating room is necessary, and a case is also assumed in which all of the nurses and the operator wear the head sets.

In addition, it is necessary that conversations between physicians and nurses in the operating room have a low delay. It is also necessary that the quality and volume of voice be appropriate for anyone who wears the head set 930 for surgery. Furthermore, it is desirable that the volume and quality of voice be individually optimized in accordance with characteristics of the voice volume and characteristics of the pronunciation of a speaker, and characteristics of the hearing ability of a listener based on age. The server 100 performs processing in accordance with the purpose at a spot where it is necessary. Inputting to the server 100 of information regarding the operator or a person who wears the head set 930 allows the server 100 to instruct on optimal processing. In a case where the voice of the speaker is low, the voice volume is increased to achieve an appropriate voice volume for the listener. In a case of a speaker whose voice is not stable in volume, the voice volume is made stable. In addition, for a listener with decreasing hearing ability, the voice volume is made higher than usual, or fine adjustments are made to the voice quality or speed for higher intelligibility.

### 10. Seventh Embodiment

In a seventh embodiment, synchronization of P in P (Picture in Picture) images is performed. The P in P images may have different delay times depending on the image processing. It is therefore desirable to display the P in P images with the times matched on the monitor outside of the operating room for which low delay is not necessary.

In the system illustrated in FIG. 19, the monitor 440 is provided outside of the operating room. In a case where the P in P images are displayed on the monitor 440 outside of the operating room, that is, in a case where a plurality of moving images are displayed in an overlaying manner on the monitor 440, processing to match the respective delay times of the moving images is performed. Unlike the monitors 410 and 420 in the operating room, there is no problem for the monitor 440 outside the operating room even if some delay occurs. Thus, the plurality of images is synchronized for higher viewability. For this purpose, under the direction of the server 100, the monitor 440 or the IP converter 800 attached to the monitor 440 monitors time signals of the plurality of images displayed on the monitor 440, and performs synchronous display. In a case where the monitor 440 or the IP converter 800 does not have such a capability, the images having undergone matching of times by the server 100 are displayed on the monitor 440.

As described above, according to each of the embodiments of the present disclosure, it is possible to perform device optimization in consideration of a plurality of devices in the operating room. This makes it possible to shorten the time for setting of the devices and thereby improve the efficiency of surgery. Consequently, the time for adjusting the surgical system 1000 is shortened and throughput of surgery is improved. Moreover, adjustments are performed by the surgical system 1000 without manual intervention. It is therefore possible to ensure uniform image quality and reduce a risk in surgery. In addition, even in a case where there is a spot not adjustable with a single device, performing adjustments in combination with other devices makes it possible to provide the operator with a better environment. That is, even in a case where adjustment of a certain single device is difficult, performing adjustment of another device makes it possible to provide a surgical environment similar to that acquired in a case where the certain single device is adjusted.

The preferred embodiments of the present disclosure have been described in detail so far with reference to the accompanying drawings; however, the technical scope of the present disclosure is not limited to such examples. It is apparent that those having ordinary skill in the art of the present disclosure are able to conceive various modifications and alterations within the scope of the technical idea of the appended claims. It should also be understood that these modifications and alterations are also within the technical scope of the present disclosure.

In addition, the effects described herein are merely illustrative and exemplary, and are non-limiting. That is, the technology according to the present disclosure may exhibit, along with or instead of the above-described effects, other effects that are apparent to those skilled in the art from the description of the present specification.

It should be noted that the following configurations are also within the technical scope of the present disclosure.

(1)

A medical device management system including:

a plurality of devices connected to a surgical network; and

an information processor configured to change settings of one or more devices out of the plurality of devices via the surgical network, in which

the information processor acquires two or more pieces of information related to the plurality of devices and changes at least one of the settings of the plurality of devices on the basis of the two or more pieces of information.

(2)

The medical device management system according to (1), in which the information processor acquires the information from output signals of the plurality of devices.

(3)

The medical device management system according to (1) or (2), in which the information processor changes the at least one of the settings of the plurality of devices on the basis of an output signal outputted from a predetermined one of the devices.

(4)

The medical device management system according to any one of (1) to (3), in which the plurality of devices includes at least any one of a monitor for surgery, a camera for surgery, a light source for surgery, a recorder that records a video image of the camera, a microphone for surgery, or a loudspeaker for surgery.

(5)

The medical device management system according to any one of (1) to (4), in which the information processor changes the at least one of the settings of the plurality of devices by changing a setting of an IP converter connected with the device.

(6)

The medical device management system according to (4), in which the information processor changes a color setting of the monitor, a setting related to a color of an image captured by the camera, or a color setting of the light source.

(7)

The medical device management system according to (4), in which the camera includes an endoscope camera, and the information processor changes a setting of the endoscope camera or the monitor on the basis of a monitor image acquired by the endoscope camera performing imaging of the monitor.

(8)

The medical device management system according to (7), in which the information processor changes at least one of settings of a plurality of the monitors for surgery on the basis of the information of at least one of the plurality of monitors for surgery.

(9)

The medical device management system according to (4), in which the information processor changes a setting of an amount of delay of at least any one of an image displayed on the monitor, an image captured by the camera, an image recorded by the recorder, a voice acquired by the microphone, or a voice outputted by the loudspeaker.

(10)

The medical device management system according to (9), in which the information processor changes the setting of the amount of delay of a plurality of images in a case where the plurality of images is displayed in an overlaying manner on the monitor.

(11)

The medical device management system according to (1), in which

the plurality of devices includes a calibration camera, and

the information processor changes the at least one of the settings of the plurality of devices on the basis of the information acquired by the calibration camera performing imaging of an output of at least any one of the plurality of devices.

(12)

The medical device management system according to (1), in which

the plurality of devices includes a calibration camera and a monitor for surgery, and

the information processor changes a setting of the monitor on the basis of the information acquired by the calibration camera performing imaging of the monitor.

(13)

The medical device management system according to any one of (1) to (12), in which the information processor determines the device to be changed in setting from among the plurality of devices on the basis of device information of the plurality of devices.

(14)

The medical device management system according to (13), in which the information processor changes the setting of the device having a wider setting adjustment range on the basis of the device information of the plurality of devices.

(15)

The medical device management system according to any one of (1) to (14), in which the information processor changes the at least one of the settings of the plurality of devices on the basis of individual information of a user who uses the surgical network.

(16)

The medical device management system according to (15), in which the information processor changes a setting of voice quality or voice volume of a loudspeaker for surgery on the basis of the individual information.

(17)

A medical device management method including:

a plurality of devices connected to a surgical network; and

changing settings of one or more devices out of the plurality of devices connected via the surgical network, wherein

the method acquires two or more pieces of information related to the plurality of devices and changes at least one of the settings of the plurality of devices on a basis of the two or more pieces of information.

### REFERENCE SIGNS LIST

100: Server
200: Operating room camera
300: Endoscope system
400, 410, 420, and 440: Monitor
500: Light source
600: Recorder
700: Shadowless lamp

1. A medical device management system comprising:
a plurality of devices connected to a surgical network; and
an information processor configured to change settings of one or more devices out of the plurality of devices via the surgical network, wherein
the information processor acquires two or more pieces of information related to the plurality of devices and changes at least one of the settings of the plurality of devices on a basis of the two or more pieces of information.

2. The medical device management system according to claim 1, wherein the information processor acquires the information from output signals of the plurality of devices.

3. The medical device management system according to claim 1, wherein the information processor changes the at least one of the settings of the plurality of devices on a basis of an output signal outputted from a predetermined one of the devices.

4. The medical device management system according to claim 1, wherein the plurality of devices includes at least any one of a monitor for surgery, a camera for surgery, a light source for surgery, a recorder that records a video image of the camera, a microphone for surgery, or a loudspeaker for surgery.

5. The medical device management system according to claim 1, wherein the information processor changes the at least one of the settings of the plurality of devices by changing a setting of an IP converter connected with the device.

6. The medical device management system according to claim 4, wherein the information processor changes a color setting of the monitor, a setting related to a color of an image captured by the camera, or a color setting of the light source.

7. The medical device management system according to claim 4, wherein the camera comprises an endoscope camera, and the information processor changes a setting of the endoscope camera or the monitor on a basis of a monitor image acquired by the endoscope camera performing imaging of the monitor.

8. The medical device management system according to claim 7, wherein the information processor changes at least one of settings of a plurality of the monitors for surgery on a basis of the information of at least one of the plurality of monitors for surgery.

9. The medical device management system according to claim 4, wherein the information processor changes a setting of an amount of delay of at least any one of an image displayed on the monitor, an image captured by the camera, an image recorded by the recorder, a voice acquired by the microphone, or a voice outputted by the loudspeaker.

10. The medical device management system according to claim 9, wherein the information processor changes the setting of the amount of delay of a plurality of images in a case where the plurality of images is displayed in an overlaying manner on the monitor.

11. The medical device management system according to claim 1, wherein
the plurality of devices includes a calibration camera, and
the information processor changes the at least one of the settings of the plurality of devices on a basis of the information acquired by the calibration camera performing imaging of an output of at least any one of the plurality of devices.

12. The medical device management system according to claim 1, wherein
the plurality of devices includes a calibration camera and a monitor for surgery, and
the information processor changes a setting of the monitor on the basis of the information acquired by the calibration camera performing imaging of the monitor.

13. The medical device management system according to claim 1, wherein the information processor determines the device to be changed in setting from among the plurality of devices on a basis of device information of the plurality of devices.

14. The medical device management system according to claim 13, wherein the information processor changes the setting of the device having a wider setting adjustment range on the basis of the device information of the plurality of devices.

15. The medical device management system according to claim 1, wherein the information processor changes the at least one of the settings of the plurality of devices on a basis of individual information of a user who uses the surgical network.

16. The medical device management system according to claim 15, wherein the information processor changes a setting of voice quality or voice volume of a loudspeaker for surgery on the basis of the individual information.

17. A medical device management method comprising:
a plurality of devices connected to a surgical network; and
changing settings of one or more devices out of the plurality of devices connected via the surgical network, wherein
the method acquires two or more pieces of information related to the plurality of devices and changes at least one of the settings of the plurality of devices on a basis of the two or more pieces of information.
